(19) United States
(12) Patent Application Publication
(10) Pub. No.: US 2012/0265092 A1
(43) Pub. Date: Oct. 18, 2012

(54) Pendulum With Visual Indicia

(75) Inventors: Alan W. Reichow, Beaverton, OR (US); Herb Yoo, Beaverton, OR (US)

(73) Assignee: Nike, Inc., Beaverton, OR (US)

(21) Appl. No.: 13/084,932

(22) Filed: Apr. 12, 2011

(51) Int. Cl. A61B 3/02

(52) U.S. Cl. 600/558

(57) ABSTRACT

A device for training and testing dynamic visual acuity is provided. The device comprises an object that is tethered using a connector, such as a string. The object may display one or more visual indicia with a given characteristic. Examples of visual indicia may include letters, numbers, shapes, and the like. Additional exemplary characteristics of the visual indicia may include varying sizes and contrasting colors. Once tethered, the object can be put into motion, which causes the subject to use more effort in viewing and identifying the visual indicia on the device.

### CROSS-REFERENCE TO RELATED APPLICATIONS

Not applicable.

### TECHNICAL FIELD

The present invention relates to vision testing. More particularly, the present invention relates to devices used in vision testing and training.

### BACKGROUND OF THE INVENTION

Within the field of vision testing and training, tools may be used in particular to measure a subject's tracking skills. Such tools may exercise a subject's periphery vision, accommodation, and dynamic visual acuity. Current tools in the art, however, may prove too simple to effectively train or test a subject with advanced vision abilities, such as an athlete.

### BRIEF SUMMARY OF THE INVENTION

The present invention relates to a device for training and testing dynamic visual acuity. More specifically, the device comprises an object that may be tethered. The object may display one or more visual indicia that may be visually perceived by the subject. Each visual indicia may possess a given characteristic. Examples of visual indicia may include letters, numbers, shapes, and the like. Additional exemplary characteristics of the visual indicia may include the identity of the indicia (i.e., what letter, etc.), varying sizes, and contrasting colors. Once tethered, the object can be put into motion, on a trajectory and/or rotationally. The movement of the object may require the subject to track the object in order to identify the visual indicia on the device, which tests or trains the subject's dynamic visual acuity. Additionally, the movement of the object on a tether may cause different visual indicia to come into the view of the subject at different times, thereby training/testing the subject's ability to track and/or perceive different traits.

Additional objects, advantages, and novel features of the invention will be set forth in part in the description which follows, and in part will become apparent to those skilled in the art upon examination of the following, or may be learned by practice of the invention.

### BRIEF DESCRIPTION OF THE SEVERAL VIEWS OF THE DRAWING

The present invention is described in detail below with reference to the attached drawing figures, wherein:

FIGS. 1A-1C illustrate an exemplary vision training device in accordance with the present invention;

FIGS. 2A and 2B illustrate another example of a vision training device in accordance with the present invention;

FIGS. 3A and 3B illustrate a further example of a vision training device in accordance with the present invention; and

FIG. 4 illustrates a flow diagram of a method for training a subject using a vision training device, in accordance with an example of the invention.

### DETAILED DESCRIPTION OF THE INVENTION

In accordance with the present invention, a device for training and testing dynamic visual acuity is provided. More specifically, the device comprises an object that is tethered using a connector, such as a string. The object may display one or more visual indicia, and each indicia may possess one or more visual characteristics. Examples of visual indicia may include letters, numbers, shapes, and the like. Exemplary characteristics of the visual indicia may include the identity of a letter/number/shape, varying sizes, contrasting colors, different physical orientations, etc. Once tethered, the object can be put into motion. For example, the object may be swung linearly, swung circularly, and/or rotated. The movement of the object may require the subject to visually track the object, while also causing different indicia on the object to enter into the subject's view. In this way, the subject must simultaneously track the object and perceive the indicia, as well as the characteristic(s) of the indicia. Moreover, the movement of the object may cause the subject to use more effort in viewing and identifying the visual indicia on the device.

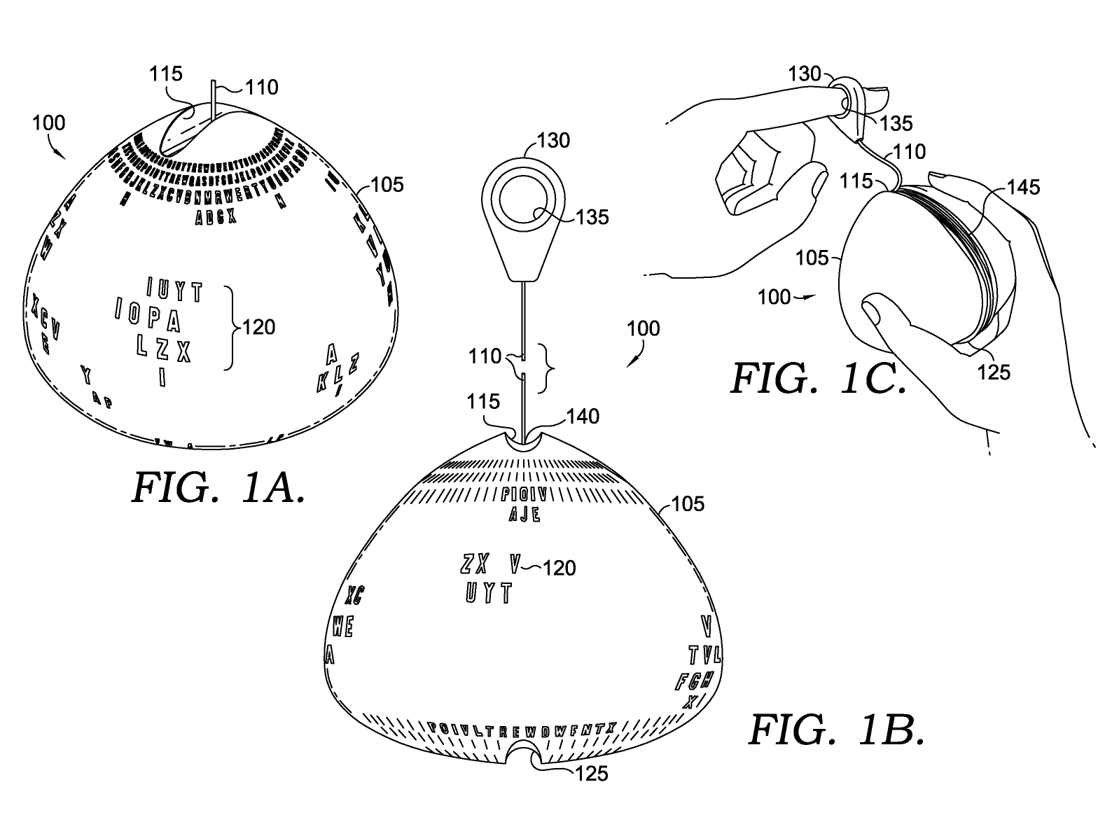

Turning now to FIGS. 1A-1C, an exemplary vision training and testing device 100 is shown. From a side view, FIG. 1A illustrates the device 100 with an object 105 and a tether 110. The object 105 may comprise of any material that is capable of being labeled or marked with indicia (for example, indicia 120). The tether 110 may consist of any material suitable to tethering the object 105. In FIG. 1B, the top angle view of the device 100 is shown. As illustrated in this example, the object 105 may have a groove 115 at the top of the object 105, where the tether 110 attaches to the object 105 at a tethering attachment point 140.

A plurality of visual indicia is indicated at 120. Indicia 120 may be displayed on the object 105 in any manner necessary to visual training. Indicia 120 may be a dot, a letter, number, shape, etc. The indicia 120 may be organized in horizontal and vertical rows. However, any variation of organization of indicia 120 may be used. For example, the rows may be evenly or unevenly spaced. In an additional example, the indicia 120 may be of a uniform size or may be of varying sizes. Further, the indicia 120 do not need to be the same type. For example, letters and numbers may both be used as indicia. Regardless of the type or combination of types of indicia 120, the characteristics of each indicia 120 may be uniform or varying. Exemplary characteristics include size, color, contrast, brightness, etc. Thus, one row of indicia may be a first color, and a second row of indicia may be a second color, etc.

In FIG. 1B, a side view of the device 100 is shown. Also illustrated is an attachment 130 that may be used to tether the object 105 via the tether 110. The attachment 130 may be of various sizes or shapes. As illustrated in FIG. 1B, the attachment 130 includes an opening 135, but, as will be shown and discussed in later figures, the attachment 135 may also include a button or other type of control mechanism to control the tether 110. The tether 110 attaches to the object at an attachment point 140. This attachment point 140 resides inside of the groove 115 at the top of the base 105. Additionally, as can be seen in this figure, a bottom groove 125 is shown directly opposing the top groove 115, at the bottom of the object 105. In FIG. 1C, the tether 110 is shown wrapped around the object 105, where the tether 110 is guided through the top groove 115 and bottom groove 125. This illustration shows the vision testing device 100 as it is stored and not in use. By providing grooves 115 and 125 to guide the wrapped tether 110, one may more easily transport and store the tether 110, thereby avoiding knotting or tangling of the tether 110 and allowing for easier use when the device is used again.

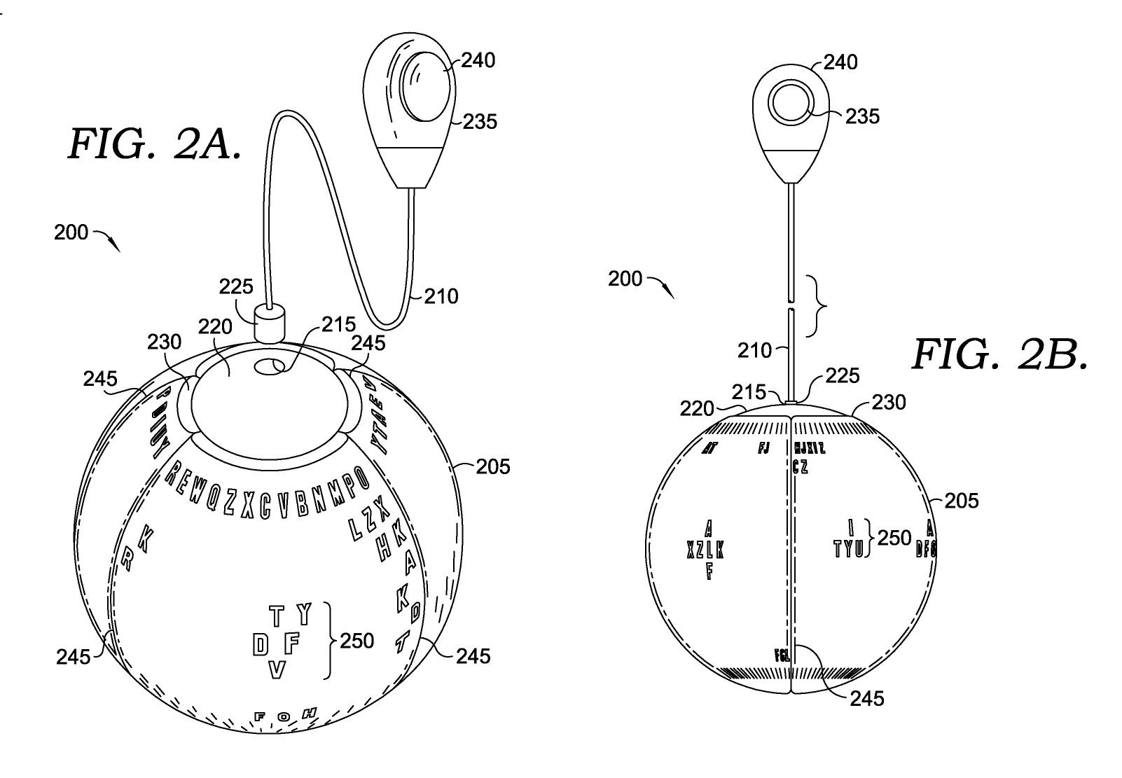

Turning now to FIGS. 2A-2B, another example of the vision training and testing device 200 in accordance with the present invention is illustrated. FIG. 2A shows a top angle view of the device 200, and FIG. 2B shows a side view of device 200, which comprises generally of an object 205, a tether 210, and an attachment 235. In this illustrated example, the object 205 comprises a base piece 220, that is in the general shape of the device 200, and a cover piece 230, that fits over the base piece 220. The base piece 220 may be comprised of any suitable material as is known in the art. The cover piece 230 may be constructed of any flexible material or fabric that is capable of stretching over the base piece 220. Exemplary materials that may be used for cover piece 230 include 2 mm Neoprene II. Further, the plurality of indicia 250 may be displayed on the cover piece 230, and thus the material of the cover piece 230 is capable of being printed or affixed with visual indicia 250. Note that this permits different indicia/characteristics to be used for training/testing for different training/testing iterations by simply changing cover piece 230. In this example, the cover piece 230 comprises four sections that are then connected at seams 245. Although the seams 245 are not necessary, they may allow the cover piece 230 to be constructed in such a manner as to fit tightly over the contours of the base piece 220.

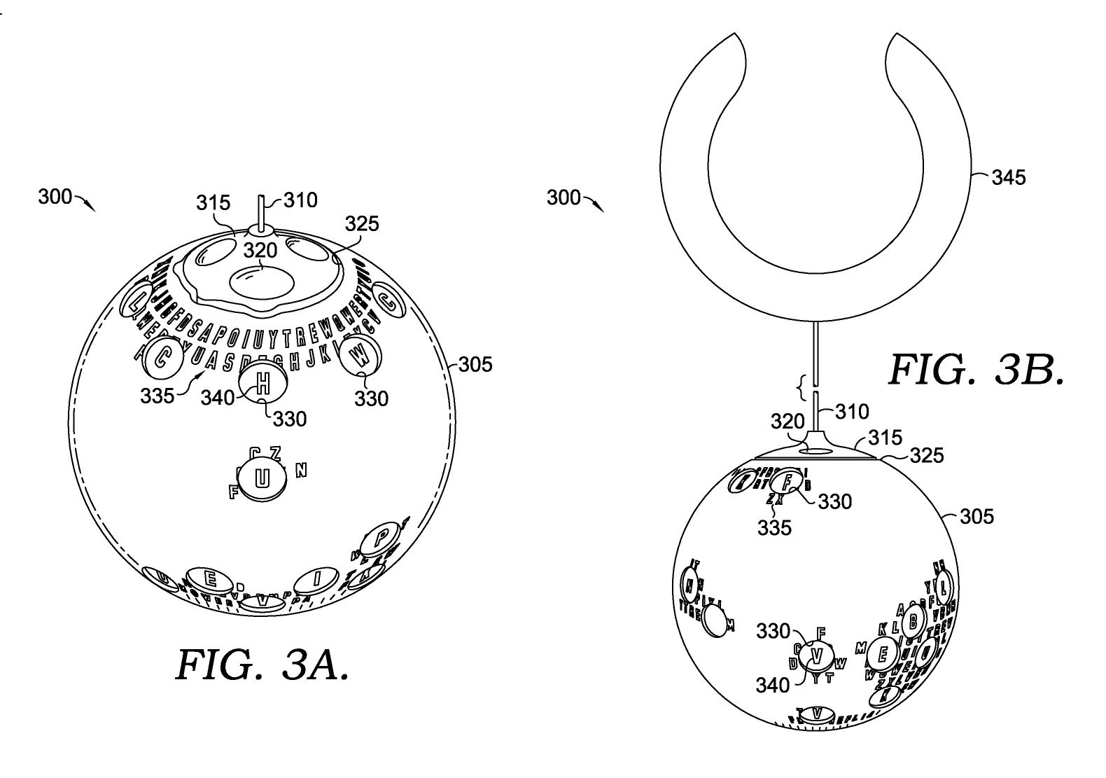

The tether 210 is connected using connecting piece 225 at the tethering attachment point 215. The connecting piece 225 allows the tether 210 to retract into an internal cavity of the object 205. The act of retracting may be initiated by a mechanism in the attachment 235, such as button 235. With a retractable tether 210, a person using the device 200 may store it more easily without tangling the tether 210. Such a retracting feature may be used with other examples of the device 200, such as the device 100 in FIGS. 1A-1C and device 300 in FIGS. 3A and 3B.

Turning now to FIGS. 3A-3B, a vision training and/or testing device 300 is illustrated in accordance with another example of the invention. The device 300 may comprise generally of an object 305, a tether 310, and an attachment 345. As in FIGS. 2A-2B, the object 305 of the device 300 is constructed of more than one piece or component. For instance, a base piece 315 may serve as a core piece of base component 305. A shell piece 325 may fit on top of the base piece 315. The base piece 315 may include a mechanism to rotate the base piece 315 in respect to the shell piece 325. In FIG. 3A, the base piece 315 is illustrated with a mechanism or a dial at the top of the device, which also may includes indentations (e.g., indentation 320) that can be used to turn the base piece 315.

Additionally, the base piece 315 and the shell piece 325 may include a plurality of visual indicia, which are indicated by 340 and 335, respectively. The visual indicia on each piece may be the same or may differ. Further, the shell piece 325 may include openings, holes, etc. that allow the visual indicia 340 on the base piece 315 to be viewed. These openings 330 may be evenly spaced over the shell piece 325, or may be spaced in a particular pattern. The base piece 315 may display indicia in a specific pattern such that different indicia or even no indicia will be displayed, depending on the rotation of the pieces with respect to each other. Providing various views of the indicia on both the base piece 315 and the shell piece 325 allows for a subject to have varying degrees of difficulty when being tested or trained using vision training and/or testing device 300.

Attachment 345 may connected to the tether 310, and tether 310 may be used to affix the device to a stationary location, such as a ceiling or wall. Attachment 345 may be constructed with an outlying groove, which may be used in winding the tether 320 for storage.

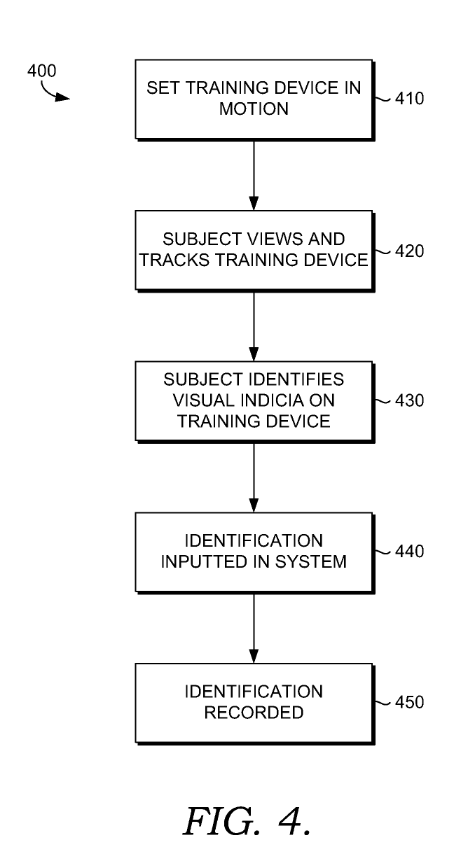

Turning now to FIG. 4, a method 400 for training a subject using a visual training device in accordance with an embodiment of the invention is illustrated. As indicated at block 410, a training device is set into motion, where the training device includes an object with a plurality of visual indicia displayed thereon. This step may be initiated by a person other than the testing subject (e.g., a trainer or other personnel). This step may be preceded by securing a tether of the training device to permit the training device to swing. In some examples, the training device may be motorized, causing the device to swing and/or rotate by itself, which allows the testing subject to self-train using such a device. At block 420, the subject views the training device and tracks the device within his or her field of view. The subject then identifies the plurality of visual indicia displayed on the training device at block 430. For example, the subject may verbally state the visual indicia, which may then be received by a trainer. Or, in a further example, the subject may use an input device, such as a joystick, to identify the visual indicia. This is shown at block 440. The identification of the visual indicia may be directly inputted in a system and recorded (e.g., at block 450). Or, it may be received by a trainer, who then inputs the information into the system.

From the foregoing, it will be seen that this invention is one well adapted to attain all the ends and objects hereinabove set forth together with other advantages which are obvious and which are inherent to the structure.

It will be understood that certain features and subcombinations are of utility and may be employed without reference to other features and subcombinations. This is contemplated by and is within the scope of the claims.

Since many possible embodiments may be made of the invention without departing from the scope thereof, it is to be understood that all matter herein set forth or shown in the accompanying drawings is to be interpreted as illustrative and not in a limiting sense.

Having thus described the invention, what is claimed is:

1. A tethered object capable of being put into motion, wherein the object comprises:
an object that swings on a tether; and
a removable cover that affixes to the object, wherein the cover displays a plurality of visual indicia with characteristics used to test vision when the object is put into motion.

2. The tethered object of claim 1, wherein the visual indicia have more than one characteristic.

3. The tethered object of claim 1, wherein the shape of the object caused the displayed visual indicia to be viewed by a subject from more than one viewing angle.

4. The tethered object of claim 1, wherein the object includes a groove at the tether attachment point and a groove at a point directly opposing the tether attachment point, and wherein the tether wraps around the object within each groove when the device is not in use.

5. A tethered object comprising:
an object having at least one visual indicia displayed thereon; and
an outer component that rotates having a plurality of visual indicia displayed thereon,
wherein the outer component includes at least one opening to permit viewing a visual indicia from the object when aligned.

6. The tethered object of claim 5, wherein the visual indicia have more than one characteristic.

7. The tethered object of claim 1, wherein the shape of the object caused the displayed visual indicia to be viewed by a subject from more than one viewing angle.

8. The tethered object of claim 1, wherein the object includes a groove at the tether attachment point and a groove at a point directly opposing the tether attachment point, and wherein the tether wraps around the object within each groove when the device is not in use.

9. A tethered object, having a first axis extending from a tether attachment point, wherein the object displays visual indicia, and wherein the shape of the object caused the displayed visual indicia to be viewed by a subject from more than one viewing angle.

10. A vision testing device testing the dynamic visual acuity of a subject, the device comprising:
an object component displaying a plurality of visual indicia with characteristics thereon; and
a retractable tether connected to the object component.

11. The vision testing device of claim 10, wherein the visual indicia have more than one characteristic.

12. A vision testing device comprising:
an object displaying a plurality of visual indicia with characteristics thereon; and
a tether connected to the object at a tether attachment point,
wherein the object includes a groove at the tether attachment point and a groove at a point directly opposing the tether attachment point, and wherein the tether wraps around the object within each groove when the device is not in use.

13. A method of training a subject, using a visual training device, wherein the training device displays a plurality of visual indicia with characteristics thereon, the method comprising:
setting the training device into motion within the field of view of the subject, wherein the subject identifies the plurality of visual indicia;
receiving the identified plurality of visual indicia; and
recording the received plurality of visual indicia.
